(19) United States
(12) Patent Application Publication
(10) Pub. No.: US 2008/0216831 A1
(43) Pub. Date: Sep. 11, 2008

(54) STANDALONE CPAP DEVICE AND METHOD OF USING

(76) Inventors: William J. McGinnis, Cincinnati, OH (US); Scott A. Metrick, Glenview, IL (US)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(21) Appl. No.: 11/683,633

(22) Filed: Mar. 8, 2007

(51) Int. Cl. A61M 16/10

(52) U.S. Cl. 128/204.21; 128/204.18; 128/205.12

(57) ABSTRACT

A stand along continuous pressure airwave pressure (CPAP) device and associated method are described for use in treating and/or diagnosing sleep apnea disorders. The CPAP device includes the interconnected components of a mouthpiece, a housing, an airway, a blower, a cover, a control circuit and an on/off switch. The method includes the steps of allowing, attaching, biting, coupling, detaching, downloading, moisturizing, obtaining, pressing, sleeping, and withdrawing.

### FIELD OF THE INVENTION

The present invention relates to treatment and/or diagnosis of partial or complete upper airway occlusion. More particularly to a stand along continuous pressure airwave pressure (CPAP) device and associated method of using the CPAP device for use in treating and/or diagnosing sleep apnea disorders.

### BACKGROUND OF THE INVENTION

Sleep apnea is a syndrome where a person stops breathing during sleep. When the airflow ceases for more than 10 seconds it is called “apnea”. Apneas can lead to decreased blood oxygenation and can often disrupt sleep. Apneas can be categorized as either central apneas, where there is no respiratory effort, or as obstructive apneas, where there is respiratory effort. In some central apneas and all obstructive apneas, the airway becomes completely closed. This closure usually occurs at the level of the tongue or soft palate. Finally, the airway may also be only partially obstructed which can also lead to decreased ventilation (hypopnea) and decreased blood oxygenation, as well as, disturbed sleep.

A treatment of sleep apnea is the administration of Continuous Positive Airway Pressure (CPAP). It is thought that the CPAP treatment acts as a pneumatic splint of the airway by the provision of a positive pressure. The ancillary air is often supplied to the airway by a motor driven blower whose outlet passes via an air delivery hose to a nose (or nose and/or mouth) mask sealingly engaged to a patient's face. An exhaust port is often provided somewhere along the delivery tube proximate to the mask. The mask is often either a nose and/or face mask or nasal prongs, pillows or cannulae.

Sometimes the CPAP device ends up warming the air through the compression in the fan and that the relative humidity of the air is consequently reduced. This dry air created by CPAP can sometimes lead to irritating the mucous membranes of the respiratory passages by desiccation.

While presently known CPAP techniques fulfill many of their respective objectives and requirements, the no CPAP techniques or devices describe a CPAP device having the interconnected components of a mouthpiece, a housing, an airway, a blower, a cover, a control circuit and an on/off switch for treating and/or diagnosing sleep apnea disorders. This combination of interconnected elements would specifically match the user's particular individual needs of making it possible to provide a stand alone means for treating and/or diagnosing sleep apnea disorders in a convenient manner.

Therefore, a need exists for a new and improved CPAP device and an associated method of using this new and improved CPAP device for treating and/or diagnosing sleep apnea. In this respect, the CPAP device according to the present invention substantially departs from the conventional concepts and designs of the prior art, and in doing so provides an apparatus primarily developed for the purpose of providing a convenient means for making it possible to treat and/or diagnose sleep apnea which is a stand alone CPAP device.

### SUMMARY OF THE INVENTION

The present continuous pressure airwave pressure (CPAP) device and method of using, according to the principles of the present invention, overcomes a number of the shortcomings of the prior art by providing a novel CPAP device for use in treating and/or diagnosing sleep apnea disorders. The CPAP device includes the interconnected components of a mouthpiece, a housing, an airway, a blower, a cover, a control circuit and an on/off switch. The method includes the steps of allowing, attaching, biting, coupling, detaching, downloading, moisturizing, obtaining, pressing, sleeping, and withdrawing.

In view of the foregoing disadvantages inherent in the known type CPAP devices now present in the prior art, the present invention provides an improved CPAP device, which will be described subsequently in great detail, is to provide a new and improved CPAP device which is not anticipated, rendered obvious, suggested, or even implied by the prior art, either alone or in any combination thereof.

To attain this, the present invention essentially comprises the interconnected components of a mouthpiece, a housing, an airway, a blower, a cover, a control circuit and an on/off switch. The method includes the steps of allowing, attaching, biting, coupling, detaching, downloading, moisturizing, obtaining, pressing, sleeping, and withdrawing.

There has thus been outlined, rather broadly, the more important features of the invention in order that the detailed description thereof that follows may be better understood, and in order that the present contribution of the art may be better appreciated.

The invention may also include any number of optional components such as humidifier chamber, a cartridge, and an air filter.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved CPAP device that has many of the advantages of the prior CPAP device and minimizing a number of their disadvantages.

It is another aspect of the present invention to provide a new and improved CPAP device that may be easily and efficiently manufactured and marketed.

An even further aspect of the present invention is to provide a new and improved CPAP device that has a low cost of manufacture with regard to both materials and labor, and which accordingly is then susceptible of low prices of sale to the consuming public, thereby making CPAP economically available to the buying public.

Still another aspect of the present invention is to provide a CPAP device that provides in the apparatuses and methods of the prior art some of the advantages thererof, while simultaneously overcoming some of the disadvantages normally associated therewith.

Even still another aspect of the present invention is to provide a CPAP device having interconnected components of a mouthpiece, a housing, an airway, a blower, a cover, a control circuit and an on/off switch so that the device can be used treating and/or diagnosing sleep apnea disorders.

Lastly, it is an object of the present invention to provide a new and improved method of using comprising the steps of using the CPAP device includes the steps of allowing, attaching, biting, coupling, detaching, downloading, moisturizing, obtaining, pressing, sleeping, and withdrawing.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of an embodiment of the CPAP device constructed in accordance with the principles of the present invention;

FIG. 2 is an exploded view of the CPAP device of the present invention; and

FIG. 3 is a frontal view of the CPAP device of the present invention.

The same reference numerals refer to the same parts throughout the various figures.

### DETAILED DESCRIPTION OF THE PREFERRED EMBODIMENT

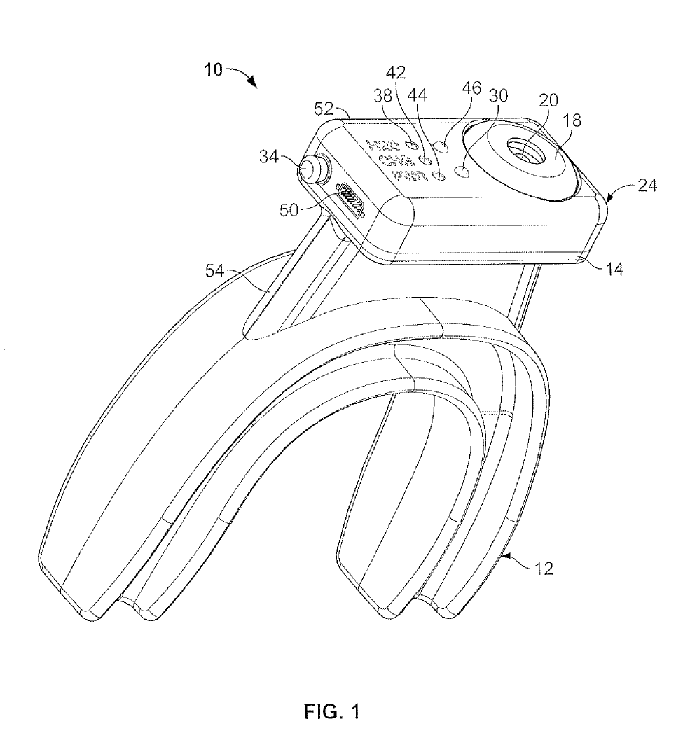

Referring now to the drawings, and in particular FIGS. 1 to 3 thereof, one preferred embodiment of the present invention is shown and generally designated by the reference numeral 10. One preferred embodiment of a continuous positive airway pressure (CPAP) device 10 comprises a mouthpiece 12, a housing 14, an airway 16, a blower 18, a cover 24, a power unit 26, a control circuit 28, and an on/off switch 30. The housing 14 is attached to the mouthpiece 12. The airway 16 is attached to the mouthpiece 12 and to the housing 14 in which the airway 16 traverses through the mouthpiece 12 and the housing 14. The blower 18 is attached to the housing 14 in which the blower 18 has an inlet 20 and an outlet 22. The inlet 20 of the blower 18 is configured to be in fluid communications with the outlet 22 of the blower 18, as well as, the outlet 22 and inlet 20 of the blower 18 are configured to be in fluid communications with the airway 16. The cover 24 is attached to the housing 14. The power unit 26 is attached to the housing 14 in which the power unit 26 is operatively coupled to the blower 18. The control circuit 28 is attached to the housing 14 in which the control circuit 28 is operatively coupled to the power unit 26 and to the blower 18. The on/off switch 30 is attached to the cover 24 in which the on/off switch 30 is operatively coupled to the control circuit 28.

An optional humidifier chamber 32 and a cartridge 34 may be added to the device 10. The optional humidifier chamber 32 is attached to the housing 14 and to the blower 18 in which the humidifier chamber 32 is in fluid communications with the airway 16 and the outlet 22 of the blower 18. The optional cartridge 34 is attached to the humidifier chamber 32 in which the optional cartridge 34 is in fluid communications with the airway 16.

An optional air filter 36 may be added to the device 10 in which the optional air filter 36 is attached to the blower 18 wherein the air filter 36 is configured to be in fluid communications with the airway 16.

An optional light emitting diode (LED) 38 may be added to the device 10 in which the light emitting diode (LED) 38 is attached to the cover 24, wherein the LED 38 is operatively coupled to the control circuit 28. The optional LED 38 may be operatively coupled to the control circuit 28 for indicating conditions selected from the group consisting of power charging conditions (CHG), on/off power conditions (PWR), and moisture conditions (H2O).

An optional detector 40 may be attached the airway 16 in which the detector 40 being operatively coupled to the control circuit 28. The optional detector 40 may be selected from the group consisting of a humidity detector 40, a temperature detector 40, a pressure detector 40, and an air flow detector 40.

An optional speaker 42 may be added to the device 10 in which the in which the optional speaker 42 may be attached to the cover 24 wherein the speaker 42 is operatively coupled to the control circuit 28.

An optional microphone 44 may be added to the device 10 in which the in which the optional microphone 44 may be attached to the cover 24 wherein the microphone 44 is operatively coupled to the control circuit 28.

An optional speaker 42 may be added to the device 10 in which the in which the optional speaker 42 may be attached to the cover 24 wherein the speaker 42 is operatively coupled to the control circuit 28.

An optional combination of a speaker 42 and a microphone 44 may be added to the device 10 in which the control circuit 28 is operatively configured to input an electrical input from the microphone 44 and to output an electrical output to the speaker 42 to drive the speaker 42 to produce noise cancellation functions in response to snoring noises. Alternately, the control circuit 28 may be operatively configured to input an electrical input from the microphone 44 and to output an electrical output to the speaker 42 to drive the speaker 42 to produce siren alarm sounds in response to snoring noises. Yet another configuration of the control circuit 28 is that it is operatively configured to output an electrical output to the speaker 42 to drive the speaker 42 to produce a musical melody.

An optional electrical socket 46 may be added to the device 10 in which the optional electrical socket 46 is attached to the cover 24 and operatively coupled to the control circuit 28 wherein the electrical socket 46 for charging the power supply.

An optional flash memory 46 may be added to the device 10 in which the optional flash memory 48 is attached to the housing 14 wherein the flash memory 48 is operatively attached to the control circuit 28.

An optional parallel interface 50 may be added to the device in which the optional parallel interface 50 is attached to the cover 24 and is operatively coupled to the control circuit 28.

An optional heat pump 52 may be added to the device 10 in which the optional heat pump 52 is attached to the cover 24 and is operatively coupled to the control circuit 28. The optional heat pump 52 may be any commercially available heat pump such as a Peltier heat pump 52.

An optional exhaust valve 54 may be added to the device 10 in which the optional exhaust valve 54 is attached to the housing 14 wherein the exhaust valve 54 in fluid communications with the airway 16.

Another preferred embodiment of the continuous positive airway pressure (CPAP) device 10 comprises a mouthpiece 12, a housing 14, an airway 16, a blower 18, a cover 24, a power unit 26, a control circuit 28, an on/off switch 30, a humidifier chamber 32, a cartridge 34, an air filter 36, at least one light emitting diode (LED) 38, a detector 40, a speaker 42, a microphone 44, an electrical socket 46, a flash memory 48, a parallel interface 50, a heat pump 52, and an exhaust valve 54. The housing 14 is attached to the mouthpiece 12. The airway 16 is attached to the mouthpiece 12 and to the housing 14 in which the airway 16 traverses through the mouthpiece 12 and the housing 14. The blower 18 is attached to the housing 14 in which the blower 18 has an inlet 20 and an outlet 22. The inlet 20 of the blower 18 being in fluid communications with the outlet 22 of the blower 18. The outlet 22 and inlet 20 of the blower 18 are in fluid communications with the airway 1 6. The cover 24 is attached to the housing 14. The power unit 26 is attached to the housing 14, the power unit 26 operatively coupled to the blower 18. The control circuit 28 is attached to the housing 14 in which the control circuit 28 is operatively coupled to the power unit 26 and to the blower 18. The on/off switch 30 is attached to the cover 24 in which the on/off switch 30 is operatively coupled to the control circuit 28. The humidifier chamber 32 is attached to the housing 14 and to the blower 18 in which the humidifier chamber 32 is in fluid communications with the airway 16 and the outlet 22 of the blower 18. The cartridge 34 is attached to the humidifier chamber 32 in which the cartridge 34 is in fluid communications with the airway 16. The air filter 36 is attached to the blower 18 in which the air filter 36 is in fluid communications with the airway 16. At least one light emitting diode (LED) 38 is attached to the cover 24 in which the LED 38 is operatively coupled to the control circuit 28. The LED 38 is operatively coupled to the control circuit 28 for indicating conditions selected from the group consisting of power charging conditions (CHG), on/off power conditions (PWR), and moisture conditions (H2O). The detector 40 is attached the airway 16 wherein the detector 40 being operatively coupled to the control circuit 28 in which the detector 40 is selected from the group consisting of a humidity detector 40, a temperature detector 40, a pressure detector 40, and an air flow detector 40. The speaker 42 is attached to the cover 24 in which the speaker 42 operatively coupled to the control circuit 28. The control circuit 28 is operatively configured to output an electrical output to the speaker 42 to drive the speaker 42 to produce a musical melody. The microphone 44 is attached to the cover 24 in which the microphone 44 is operatively coupled to the control circuit 28. The electrical socket 46 is attached to the cover 24 and is operatively coupled to the control circuit 28 wherein the electrical socket 46 is for charging the power supply. The flash memory 48 is attached to the housing 14 in which the flash memory 48 is operatively attached to the control circuit 28. The parallel interface 50 is attached to the cover 24 and is operatively coupled to the control circuit 28. The heat pump 52 is attached to the cover 24 and is operatively coupled to the control circuit 28. The exhaust valve 54 is attached to the housing 14 in which the exhaust valve 54 is in fluid communications with the airway 16.

On preferred embodiment of a method of using a continuous positive airway pressure (CPAP) device 10 comprises the steps of: allowing, attaching, biting, coupling, detaching, downloading, moisturizing, obtaining, pressing, sleeping, and withdrawing. The obtaining step comprises obtaining the device 10 comprising: a mouthpiece 12; a housing 14 attached to the mouthpiece 12; a airway 16 attached to the mouthpiece 12 and the housing 14, the airway 16 defining an airway 16 traversing through the mouthpiece 12 and the housing 14; a blower 18 attached to the housing 14, the blower 18 having an inlet 20 and an outlet 22, the inlet 20 of the blower 18 being in fluid communications with the outlet 22 of the blower 18, the outlet 22 and inlet 20 of the blower 18 in fluid communications with the airway 16; a cover 24 attached to the housing 14; a power unit 26 attached to the housing 14, the power unit 26 operatively coupled to the blower 18; a control circuit 28 attached to the housing 14, the control circuit 28 operatively coupled to the power unit 26 and to the blower 18; an on/off switch 30 attached to the cover 24, the on/off switch 30 operatively coupled to the control circuit 28; a humidifier chamber 32 attached to the housing 14 and to the blower 18, wherein the humidifier chamber 32 in fluid communications with the airway 16 and the outlet 22 of the blower 18; a cartridge 34 attached to the humidifier chamber 32, the cartridge 34 in fluid communications with the airway 16; an air filter 36 attached to the blower 18 wherein the air filter 36 in fluid communications with the airway 16, at least one light emitting diode (LED) 38 attached to the cover 24, the LED 38 operatively coupled to the control circuit 28 wherein the LED 38 operatively coupled to the control circuit 28 for indicating conditions selected from the group consisting of power charging conditions (CHG), on/off power conditions (PWR), and moisture conditions (H2O); a detector 40 attached the airway 16 wherein the detector 40 being operatively coupled to the control circuit 28 wherein the detector 40 is selected from the group consisting of a humidity detector 40, a temperature detector 40, a pressure detector 40, and an air flow detector 40; a speaker 42 attached to the cover 24, the speaker 42 operatively coupled to the control circuit 28 wherein the control circuit 28 is operatively configured to output an electrical output to the speaker 42 to drive the speaker 42 to produce a musical melody; a microphone 44 attached to the cover 24 wherein the microphone 44 is operatively coupled to the control circuit 28; an electrical socket 46 attached to the cover 24 and operatively coupled to the control circuit 28, the electrical socket 46 for charging the power supply; a flash memory 48 attached to the housing 14, the flash memory 48 operatively attached to the control circuit 28; a parallel interface 50 attached to the cover 24 and operatively coupled to the control circuit 28; a heat pump 52 attached to the cover 24 and operatively coupled to the control circuit 28; and an exhaust valve 54 attached to the housing 14, the exhaust valve 54 in fluid communications with the airway 16. The biting step comprises biting onto the mouthpiece 12 to mount the device 10 in a mouth of a user. The pressing step comprises pressing onto the on/off switch 30 to activate the control circuit 28. The detaching step comprises detaching the cartridge 34 from the humidifier chamber 32. The moisturizing step comprises moisturizing the detached cartridge 34. The attaching step comprises attaching the moistened cartridge 34 to the humidifier chamber 32. The sleeping step comprises sleeping with the device 10 mounted in the mouth of the user. The allowing step comprises allowing the control circuit 28 to store sleep response data into the flash memory 48. The withdrawing step comprises withdrawing the mouthpiece 12 from the mouth of a user. The coupling step comprises coupling the parallel interface 50 to a computer. The downloading step comprises downloading the sleep response data stored in the flash memory 48

Referring now to FIG. 1 which depicts a perspective view of an CPAP device 10 showing the mouthpiece 12, the housing 14, the blower 18, the cover 24, the on/off switch 30, the cartridge 34, the air filter 36, the LED 38, the speaker 42, the microphone 44, the electrical socket 46, the parallel interface 50, the heat pump 52, and the exhaust valve 54. The housing 14 is shown attached to the mouthpiece 12. The blower 18 is shown attached to the housing 14 with an inlet 20. The cover 24 is shown attached to the housing 14, The on/off switch 30 is shown attached to the housing 14. The cartridge 34 is shown mounted within the cover 24. The LED 38 is shown attached to the cover 24 The speaker 42 is shown attached to the cover 24. The microphone 44 is shown attached to the cover 24. The electrical socket 46 is shown attached to the cover 24. The parallel interface 50 is shown attached to the cover 24. The heat pump 52 is shown attached to the cover 24. The exhaust valve 54 is shown attached to the housing 14.

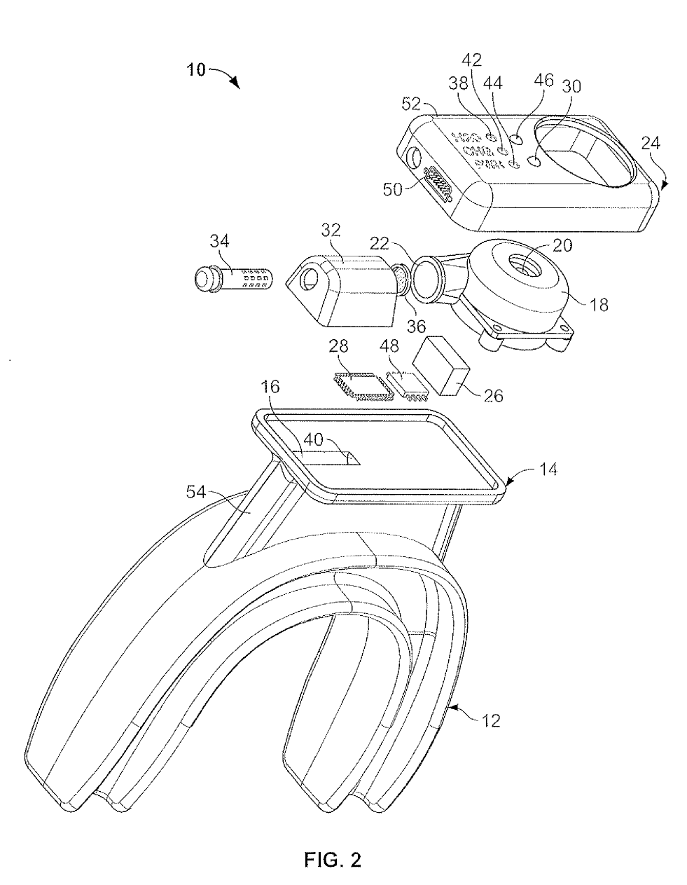

Referring now to FIG. 2 which depicts an exploded perspective view of one embodiment of the CPAP device 10 showing the mouthpiece 12, the housing 14, the airway 16, the blower 18, the cover 24, the power unit 26, the control circuit 28, the on/off switch 30, the humidifier chamber 32, the cartridge 34, the air filter 36, the LED 38, the detector 40, the speaker 42, the microphone 44, the electrical socket 46, the flash memory 48, the parallel interface 50, the heat pump 52, and the exhaust valve 54. The housing 14 is shown attached to the mouthpiece 12. The airway 16 is shown attached to the housing 14 in which the airway 16 traverses through the mouthpiece 12 and the housing 14. The blower 18 is shown having an inlet 20 and an outlet 22. The on/off switch 30 is shown attached to the cover 24. The LED 38 is shown attached to the cover 24. The detector 40 is shown attached the airway 16. The speaker 42 is shown attached to the cover 24. The microphone 44 is shown attached to the cover 24. The electrical socket 46 is shown attached to the cover 24. The flash memory 48 is shown. The parallel interface 50 is shown attached to the cover 24. The heat pump 52 is shown attached to the cover 24. The exhaust valve 54 is shown attached to the housing 14 in which the exhaust valve 54 is in fluid communications with the airway 16.

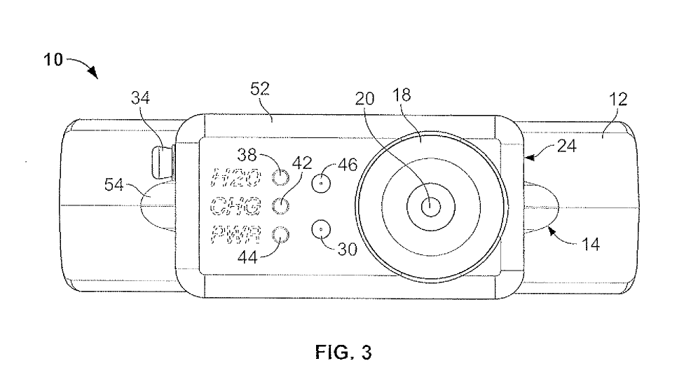

Referring now to FIG. 3 which depicts a front plan view of the CPAP device 10 showing the mouthpiece 12, the housing 14, the blower 18, the cover 24, the on/off switch 30, the cartridge 34, the LED 38, the speaker 42, the microphone 44, the electrical socket 46, the heat pump 52, and the exhaust valve 54. The housing 14 is shown attached to the mouthpiece 12. The blower 18 is shown having an inlet 20. The on/off switch 30 is attached to the cover 24. The LED 38 is shown attached to the cover 24. The speaker 42 is shown attached to the cover 24. The microphone 44 is shown attached to the cover 24. The electrical socket 46 is shown attached to the cover 24. The heat pump 52 is shown attached to the cover 24. The exhaust valve 54 is shown attached to the housing 14.

As to the manner of usage and operation of the present invention, the same should be apparent from the above description. Accordingly, no further discussion relating to the manner of usage and operation will be provided.

While a preferred embodiment of the CPAP device has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, failing within the scope of the invention,

It is claimed:

1. A continuous positive airway pressure (CPAP) device comprising
a mouthpiece;
a housing attached to the mouthpiece;
an airway attached to the mouthpiece and the housing wherein the airway traversing through the mouthpiece and the housing;
a blower attached to the housing, the blower having an inlet and an outlet, the inlet of the blower being in fluid communications with the outlet of the blower, the outlet and inlet of the blower in fluid communications with the airway;
a cover attached to the housing;
a power unit attached to the housing, the power unit operatively coupled to the blower;
a control circuit attached to the housing, the control circuit operatively coupled to the power unit and to the blower; and
an on/off switch attached to the cover, the on/off switch operatively coupled to the control circuit.

2. The device of claim 1 further comprising
a humidifier chamber attached to the housing and to the blower wherein the humidifier chamber in fluid communications with the airway and the outlet of the blower; and
a cartridge attached to the humidifier chamber, the cartridge in fluid communications with the airway.

3. The device of claim 1 further comprising an air filter attached to the blower wherein the air filter in fluid communications with the airway.

4. The device of claim 1 further comprising at least one light emitting diode (LED) attached to the cover, the LED operatively coupled to the control circuit.

5. The device of claim 4 wherein the LED operatively coupled to the control circuit for indicating conditions selected from the group consisting of power charging conditions (CHG), on/off power conditions (PWR), and moisture conditions (H2O).

6. The device of claim 1 further comprising a detector attached the airway wherein the detector being operatively coupled to the control circuit.

7. The device of claim 7 wherein the detector is selected from the group consisting of a humidity detector, a temperature detector, a pressure detector, and an air flow detector.

8. The device of claim 1 further comprising a speaker attached to the cover wherein the speaker is operatively coupled to the control circuit.

9. The device of claim 8 further comprising a microphone attached to the cover wherein the microphone is operatively coupled to the control circuit.

10. The device of claim 9 wherein the control circuit is operatively configured to input an electrical input from the microphone and to output an electrical output to the speaker to drive the speaker to produce noise cancellation functions in response to snoring noises.

11. The device of claim 9 wherein the control circuit is operatively configured to input an electrical input from the microphone and to output an electrical output to the speaker to drive the speaker to produce siren alarm sounds in response to snoring noises.

12. The device of claim 8 wherein the control circuit is operatively configured to output an electrical output to the speaker to drive the speaker to produce a musical melody.

13. The device of claim 1 further comprising an electrical socket attached to the cover and operatively coupled to the control circuit, the electrical socket for charging the power supply.

14. The device of claim 1 further comprising a flash memory attached to the housing, the flash memory operatively attached to the control circuit.

15. The device of claim 1 further comprising a parallel interface attached to the cover and operatively coupled to the control circuit.

16. The device of claim 1 further comprising a heat pump attached to the cover and operatively coupled to the control circuit.

17. The device of claim 16 wherein the heat pump is a Pettier heat pump.

18. The device of claim 1 further comprising an exhaust valve attached to the housing, the exhaust valve in fluid communications with the airway.

19. A continuous positive airway pressure (CPAP) device comprising
a mouthpiece;
a housing attached to the mouthpiece;
a airway attached to the mouthpiece and the housing, the airway traverses through the mouthpiece and the housing;
a blower attached to the housing, the blower having an inlet and an outlet, the inlet of the blower being in fluid communications with the outlet of the blower, the outlet and inlet of the blower in fluid communications with the airway;
a cover attached to the housing;
a power unit attached to the housing, the power unit operatively coupled to the blower;
a control circuit attached to the housing, the control circuit operatively coupled to the power unit and to the blower;
an on/off switch attached to the cover, the on/off switch operatively coupled to the control circuit;
a humidifier chamber attached to the housing and to the blower, wherein the humidifier chamber in fluid communications with the airway and the outlet of the blower;
a cartridge attached to the humidifier chamber, the cartridge in fluid communications with the airway;
an air filter attached to the blower wherein the air filter in fluid communications with the airway;
at least one light emitting diode (LED) attached to the cover, the LED operatively coupled to the control circuit wherein the LED operatively coupled to the control circuit for indicating conditions selected from the group consisting of power charging conditions (CHG), on/off power conditions (PWR), and moisture conditions (H2O);
a detector attached the airway wherein the detector being operatively coupled to the control circuit wherein the detector is selected from the group consisting of a humidity detector, a temperature detector, a pressure detector, and an air flow detector;
a speaker attached to the cover, the speaker operatively coupled to the control circuit wherein the control circuit is operatively configured to output an electrical output to the speaker to drive the speaker to produce a musical melody;
a microphone attached to the cover wherein the microphone is operatively coupled to the control circuit;
an electrical socket attached to the cover and operatively coupled to the control circuit, the electrical socket for charging the power supply;
a flash memory attached to the housing, the flash memory operatively attached to the control circuit;
a parallel interface attached to the cover and operatively coupled to the control circuit;
a heat pump attached to the cover and operatively coupled to the control circuit; and
an exhaust valve attached to the housing, the exhaust valve in fluid communications with the airway.

20. A method of using a continuous positive airway pressure (CPAP) device, the method comprising the steps of:
obtaining the device comprising:
a mouthpiece;
a housing attached to the mouthpiece;
a airway attached to the mouthpiece and the housing, the airway traverses through the mouthpiece and the housing;
a blower attached to the housing, the blower having an inlet and an outlet, the inlet of the blower being in fluid communications with the outlet of the blower, the outlet and inlet of the blower in fluid communications with the airway;
a cover attached to the housing;
a power unit attached to the housing, the power unit operatively coupled to the blower;
a control circuit attached to the housing, the control circuit operatively coupled to the power unit and to the blower;
an on/off switch attached to the cover, the on/off switch operatively coupled to the control circuit;
a humidifier chamber attached to the housing and to the blower, wherein the humidifier chamber in fluid communications with the airway and the outlet of the blower;
a cartridge attached to the humidifier chamber, the cartridge in fluid communications with the airway;
an air filter attached to the blower wherein the air filter in fluid communications with the airway;
at least one light emitting diode (LED) attached to the cover, the LED operatively coupled to the control circuit wherein the LED operatively coupled to the control circuit for indicating conditions selected from the group consisting of power charging conditions (CHG), on/off power conditions (PWR), and moisture conditions (H2O);
a detector attached the airway wherein the detector being operatively coupled to the control circuit wherein the detector is selected from the group consisting of a humidity detector, a temperature detector, a pressure detector, and an air flow detector;
a speaker attached to the cover, the speaker operatively coupled to the control circuit wherein the control circuit is operatively configured to output an electrical output to the speaker to drive the speaker to produce a musical melody;
a microphone attached to the cover wherein the microphone is operatively coupled to the control circuit;
an electrical socket attached to the cover and operatively coupled to the control circuit, the electrical socket for charging the power supply;
a flash memory attached to the housing, the flash memory operatively attached to the control circuit;
a parallel interface attached to the cover and operatively coupled to the control circuits
a heat pump attached to the cover and operatively coupled to the control circuit; and
an exhaust valve attached to the housing, the exhaust valve in fluid communications with the airway;
biting onto the a mouthpiece to mount the device in a mouth of a user; pressing onto the on/off switch to activate the control circuit;
detaching the cartridge from the humidifier chamber; moisturizing the detached cartridge;
attaching the moistened cartridge to the humidifier chamber;
sleeping with the device mounted in the mouth of the user;
allowing the control circuit to store sleep response data into the flash memory;
withdrawing the mouthpiece from the mouth of a user;
coupling the parallel interface to a computer; and
downloading the sleep response data stored.
